(19) United States
(12) Patent Application Publication
(10) Pub. No.: US 2019/0154707 A1
(43) Pub. Date: May 23, 2019

(54) Smart Advisor for Blood Test Evaluation

(71) Applicant: Bio-Rad Laboratories, Inc., Hercules, CA (US)

(72) Inventors: Marco Flamini, Vallejo, CA (US); Judith Borsuk Kessler, Rehovot (IL); Anat Avidan Zipor, Rehovot (IL)

(21) Appl. No.: 16/194,185

(22) Filed: Nov. 16, 2018

Related U.S. Application Data
(60) Provisional application No. 62/587,958, filed on Nov. 17, 2017.

(51) Int. Cl. G01N 33/72; G16H 50/20; G01N 30/02

(52) U.S. Cl. CPC G01N 33/721; G16H 10/60; G01N 30/02; G16H 50/20

(57) ABSTRACT

A smart advisor receives blood test data for a sample and applies a series of rules in a pipeline to determine a match between the results from the sample and one of a set of possible presumptive patterns. The presumptive patterns each correspond to a condition. The smart advisor generates an enhanced report that identifies the selected pattern. The report may also include a likelihood that the presumptive pattern is a correct match for the sample as well as comments and notes. The comments and notes may suggest additional testing that should be performed, identify common diagnosis pitfalls, identify demographic factors that may correlate with diagnosis, suggest family studies to confirm inheritance of a variant, and alert on reproductive risks if both partners are carriers of a specific variant.

### CROSS REFERENCE TO RELATED APPLICATIONS

This application claims the benefit of U.S. Provisional Application No. 62/587,958, filed Nov. 17, 2017, which is incorporated by reference.

### BACKGROUND

### 1. Technical Field

The subject matter described generally relates to analyzing diagnostic testing data, and in particular to computer-aided blood test evaluation.

### 2. Background Information

A hemoglobinopathy is a genetic defect that results in an unusual structure of hemoglobin molecules in an individual's blood. For example, sickle-cell disease is caused by a hemoglobinopathy that can result in the red blood cells forming a rigid sickle shape under certain circumstances. These misshapen red blood cells can obstruct capillaries and restrict blood flow, leading to a range of health problems. In contrast, a thalassemia is a genetic condition that results in reduced hemoglobin production (e.g., severe anemia). Some hemoglobinopathies also impact hemoglobin production, and are thus also thalassemias.

Various medical conditions are characterized by the presence of certain hemoglobin variants and the proportions of different variants in the blood. Blood tests provide information about the proportions of different hemoglobin variants in a blood sample. However, interpreting this information can be challenging. Different conditions can have similar impacts on the presence of certain variants. The analysis is further complicated because other environmental and health factors can impact the proportions of the variants present. For example, an unusually large amount of hemoglobin F may indicate a genetic disorder or may indicate that an individual was pregnant or an infant at the time the sample was taken. Furthermore, relatively small amounts of a variant (or change in the amount of a variant present) may be clinically significant, but masked by variants that are present in far larger amounts.

Conventional approaches for analyzing blood test data rely heavily on human analysts, who may be subject to making errors and require significant time and training to reach diagnoses. Although some systems use computer-based technology to present blood test data, it is presented in forms that are not conducive to easy interpretation and diagnosis by human operators. Thus, such systems are still prone to human error and require significant amounts of operator training.

### BRIEF DESCRIPTION OF THE DRAWINGS

FIG. 1 is a high-level block diagram illustrating a networked computing environment in which diagnostic data is generated and analyzed, according to one embodiment.

FIG. 2 is a high-level block diagram illustrating a laboratory terminal suitable for use in the networked computing environment of FIG. 1, according to one embodiment.

FIG. 3 is a high-level block diagram illustrating the smart advisor of a laboratory terminal, according to one embodiment.

FIG. 4 is a high-level block diagram illustrating an example of a computer suitable for use as a laboratory terminal, according to one embodiment.

FIG. 5 illustrates an example chromatogram, according to one embodiment.

FIG. 6 is a table illustrating example pattern rules that relate specific ranges of chromatography results to presumptive patterns, according to one embodiment.

FIG. 7 illustrates an example enhanced report produced by the smart advisor, according to one embodiment.

FIG. 8 illustrates a second example enhanced report produced by the smart advisor, according to one embodiment.

FIGS. 9A and 9B are a flow-chart illustrating a method for producing an enhanced report using a smart advisor, according to one embodiment.

FIG. 10 is an interactions diagram illustrating a drill in operation to edit the comments of a pattern rule, according to one embodiment.

FIG. 11 is a flow-chart illustrating a method for generating an enhanced report that includes automatically obtaining additional test data to refine the presumptive pattern identified in the enhanced report, according to one embodiment.

FIG. 12 is a plot of example blood test data dataset for an individual who is a carrier of the sickle cell S trait.

FIG. 13 is a plot of example blood test data dataset for an individual who has sickle cell with β thalassemia.

FIG. 14 is a screenshot illustrating analysis quality rules in a rule editor, according to one embodiment.

FIG. 15 is a screenshot illustrating variant identification rules in a rule editor, according to one embodiment.

FIG. 16 is a screenshot illustrating pattern rules in a rule editor, according to one embodiment.

FIG. 17 is a screenshot illustrating result evaluation rules in a rule editor, according to one embodiment.

The figures depict various embodiments for purposes of illustration only. One skilled in the art will readily recognize from the following discussion that alternative embodiments of the structures and methods illustrated herein may be employed without departing from the principles described herein. It is noted that wherever practicable similar or like reference numbers are used in the figures to indicate similar or like functionality.

### DETAILED DESCRIPTION

Computer technology provides novel opportunities to analyze blood test data and more reliably distinguish between different causes of observed hemoglobin variant levels in a sample. As noted previously, existing systems are prone to human error and require significant training of human operators. These and other problems are addressed by a smart advisor system for blood test evaluation.

### Overview and Benefits

The smart advisor is used as part of a laboratory blood test system for identifying genetic conditions based on the relative proportions of various types of hemoglobin in a sample. The smart advisor applies a series of rules in a pipeline to determine a match between the results from the sample and one of a set of possible presumptive patterns. The presumptive patterns each correspond to a specific phenotype. The smart advisor generates an enhanced report that identifies the selected pattern and/or phenotype. The report may also include a likelihood that the presumptive pattern is a correct match for the sample as well as comments and notes. The comments and notes may suggest additional testing that should be performed, identify common diagnosis pitfalls, provide additional information about the corresponding condition (e.g., demographic factors that correlate with diagnosis), identify possible reproductive risks, and the like.

The automated application of rules has several advantages. First, it helps with result interpretation enabling laboratories to deliver more standardized results without the need for additional training. In fact, it may reduce the amount of training required for laboratory technicians to operate efficiently. Second, it enables results to be compared substantially in real time with large databases of reference cases that are available on-line, which may result in more accurate preliminary identifications of potential conditions. Third, the use of a rules pipeline enables automatic detection of possible errors or interferences in the test results at different stages of the analysis. This may allow for automated or semi-automated triggering of additional or repeat testing, increasing the reliability of the ultimate results. Fourth, the rules can result in suggestions for next steps in reaching a diagnosis, which can reduce reliance on human-made connections between test results and possible causes. In some cases, the next steps can be triggered automatically or semi-automatically (e.g., if the required data for the next step is already available in a database), reducing the time taken to complete the testing process. In sum, the smart advisor provides a user interface for analyzing blood test data that may be more efficient and/or accurate than existing approaches.

### Example Systems

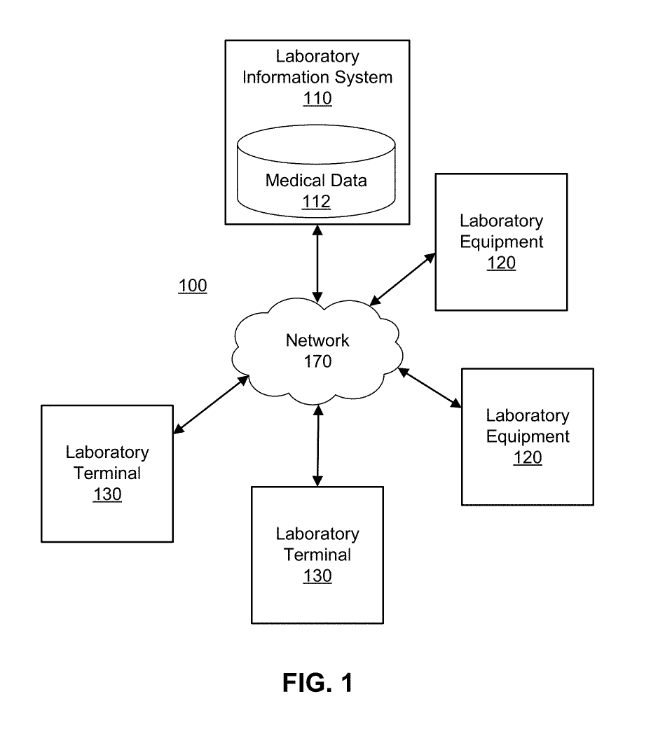

FIG. 1 shows one embodiment of a networked computing environment 100 in which diagnostic data is generated and analyzed. In the embodiment shown in FIG. 1, the networked computing environment includes a laboratory information system (LIS) 110, laboratory equipment 120, and laboratory terminals 130, all connected via a network 170. Although two items of laboratory equipment 120 and two laboratory terminals 130 are shown, a given deployment may include any amount of equipment and any number of terminals (including just a single terminal). In other embodiments, the networked computing environment 100 contains different and/or additional elements. In addition, the functions may be distributed among the elements in a different manner than described. For example, each item of laboratory equipment 120 may include a computer system that provides the functionality of a laboratory terminal 130.

The LIS 110 is a computer-based system that supports the operations of the laboratory. In various embodiments, the LIS 110 provides tools that help technicians and other users function in the laboratory efficiently. For example the LIS 110 might provide data tracking, automated backup, data exchange, work flow management, sample management, data analysis, data mining, instrument management, report generation, data auditing, and the like. In the embodiment shown in FIG. 1, the LIS 110 stores medical data 112. The medical data 112 is stored on one or more computer readable media, such as a hard drive. The medical data 112 can include patient records, test results, medical literature, and the like. One of skill in the art will recognize other functionality that the LIS 110 may provide and other types of data that may be stored as part of the medical data 112.

The laboratory equipment 120 is one or more devices that perform medical tests. In one embodiment, the laboratory equipment 120 includes a chromatography system that produces a chromatogram indicating the relative proportions of different variants of hemoglobin present in a sample. An example of such a system is the D-100™ produced by Bio-Rad™. The laboratory equipment 120 can also include devices that perform other tests, such as DNA testing, urine testing, and the like. By identifying a possible phenotype, a smart advisor may trigger a series of tests for aiding in differential diagnosis of the sample, e.g., a sickling test, a stability test (isopropanol test), electrophoresis tests, MS/MS, molecular studies, and the like.

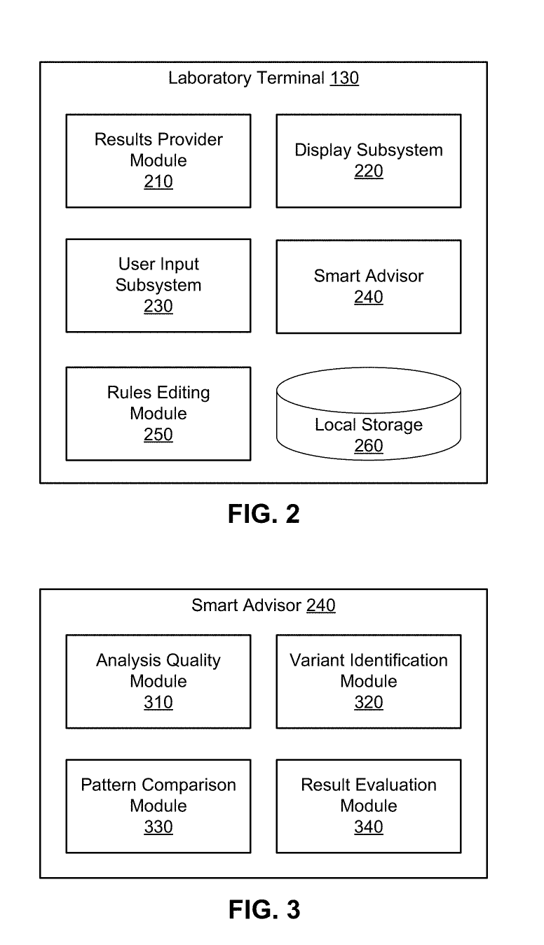

The laboratory terminals 130 are computing devices with which users interact with the LIS 110 and lab equipment 120. In various embodiments, a technician initiates a test on a sample using a terminal 130 that includes a smart advisor. The smart advisor may be software installed on laboratory terminal 130 or remote software (e.g., cloud-based software as a service) accessed via an interface on the terminal. The terminal 130 presents a report generated by the smart advisor including results analysis and suggestions. In one embodiment, the technician approves the report and it is sent to the LIS 110 for storage. In another embodiment, a laboratory supervisor must also approve the report (e.g., using a second terminal 130). The terminal 130 may also send instructions (e.g., to the LIS 110) to initiate additional tests and/or provide the results of previously conducted tests based on the recommendations generated by the smart advisor. Embodiments of the terminal 130, and in particular operation of the smart advisor, are described in additional detail below, with reference to FIGS. 2 and 3.

The network 170 provides the communication channels via which the other elements of the networked computing environment 100 communicate. The network 170 can include any combination of local area and/or wide area networks, using both wired and/or wireless communication systems. In one embodiment, the network 170 uses standard communications technologies and/or protocols. For example, the network 170 can include communication links using technologies such as Ethernet, 802.11, worldwide interoperability for microwave access (WiMAX), 3G, 4G, 5G, code division multiple access (CDMA), digital subscriber line (DSL), etc. Examples of networking protocols used for communicating via the network 170 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 170 may be represented using any suitable format, such as hypertext markup language (HTML) or extensible markup language (XML). In one embodiment, some or all of the components are connected using an RS-232 serial connection. In some embodiments, all or some of the communication links of the network 170 may be encrypted using any suitable technique or techniques.

FIG. 2 shows one embodiment of a laboratory terminal 130 suitable for use in the networked computing environment 100 of FIG. 1. In the embodiment shown in FIG. 2, the laboratory terminal 130 includes a results provider 210, a display subsystem 220, a user input subsystem 230, a smart advisor 240, a rules editing module 250, and local storage 260. In other embodiments, the laboratory terminal 130 contains different and/or additional elements. In addition, the functions may be distributed among the elements in a different manner than described. For example, in one embodiment, the rules editing module 250 is omitted, with the rules used by the smart advisor 240 being pre-programmed.

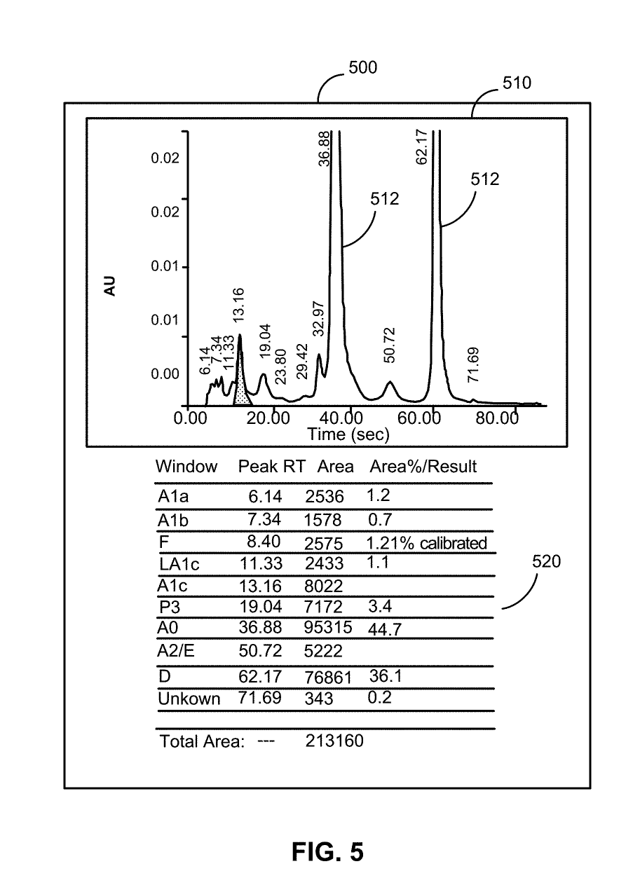

The results provider module 210 interfaces with laboratory equipment 120 to obtain medical data. In one embodiment, the medical data is blood chromatography data that the results provider module 210 uses to create a chromatogram. Alternatively, the chromatogram may be generated by the lab equipment 120 (or elsewhere in the networked computing environment 100) and provided as input to the results provider module 210. FIG. 5 shows an example of a chromatogram 500, according to one embodiment. The chromatogram 500 includes a visual representation of the data 510 and a data table 520. The visual representation 510 includes a plot of detector response over time that includes several peaks 512 (of which only two are labelled for clarity). The data table 520 identifies the retention time (i.e., the time at which the strongest detector response was observed for a peak 512) in various windows expected to correspond to different variants of hemoglobin (e.g., A1a, A1b, F, etc.). The data table 520 also includes the area of each peak 512 (which corresponds to the total amount of the given variant present in the sample) and the percentage of the total result represented by each peak.

Referring back to FIG. 2, the display subsystem 220 presents information and controls to a user (e.g., a laboratory scientist). In one embodiment, the display subsystem 220 provides controls with which a technician initiates a test by the laboratory equipment 120. The display subsystem 220 then provides controls to enable the operator to view and analyze the results of the test (e.g., using the smart advisor 240). The display subsystem 220 may also provide other functionality, such as viewing patient records, configuring the laboratory equipment 120, viewing status/maintenance data, and the like.

The user input subsystem 230 receives input from a user (e.g., a laboratory scientist or supervisor) and provides it to other elements of the terminal 130. In one embodiment, the user input subsystem 230 includes a touch screen. Controls are presented on the touch screen enabling the user to control the laboratory equipment 120 and/or interact with the smart advisor 240. Further details of embodiments of the user interface provided by the user input subsystem 230 are provided below, with reference to FIGS. 3, 8-10, and 14-17.

The smart advisor 240 analyzes the data provided by the results provider module 210 to generate a report. In various embodiments, the smart advisor 240 applies a set of customizable rules to provisionally match the data to a specific medical condition. The smart advisor 240 then generates a report that identifies the provisional match and includes comments regarding interpretation of the result. The report may additionally include a likelihood that the provisional match is correct and/or a recommendation for further testing that will allow a definitive diagnosis. For example, if the results suggest the subject may be a carrier of an inheritable blood disorder, the smart advisor 240 might recommend a DNA test for verification if the subject is considering having children. In one embodiment, the smart advisor 240 may automatically trigger further analysis if the required data and/or equipment is available and update the report accordingly. Details of various embodiments of the smart advisor 240 are described in greater detail below, with reference to FIG. 3.

The rules editing module 250 provides a user interface via which an authorized user (e.g., a systems administrator) can modify the rules used by the smart advisor 240. In one embodiment, a rule defines an input, a comparison between the input and one or more pre-determined conditions, and an output based on the result of the comparison. The input can be one or more variables, either obtained directly from the data provided the results provider module 210 or the output of other rules. The comparison indicates one or more ways in which the input should be compared to pre-determined conditions. This includes determining whether an input variable: matches a pre-determined value, is greater than a threshold, is lesser than a threshold, falls within a specified range, and the like. The output includes information regarding the result of the comparison. Many forms of output are possible, ranging from flagging a test result as possibly unreliable to indicating that one or more peaks are outside of a normal range of values, and preliminarily concluding that the sample indicates the subject has a particular condition to adding a comment to the resulting report regarding additional testing.

Because the input for a rule can be the output of another rules, the rules can be chained to perform detailed analysis. In one embodiment, a laboratory terminal 130 comes pre-programmed with default rules that users can modify and expand depending on the specific needs of the user and the specific data available from the laboratory equipment 120. The discussion below will identify several examples of rules. One of skill in the art will recognize other rules that may be used based on the identified rules.

FIG. 3 shows one embodiment of the smart advisor 240 of the laboratory terminal 120 shown in FIG. 2. In the embodiment shown in FIG. 3, the smart advisor 240 includes an analysis quality module 310, a variant identification module 320, a pattern comparison module 330, and a results evaluation module 340. In other embodiments, the smart advisor 240 contains different and/or additional elements. In addition, the functions may be distributed among the elements in a different manner than described.

The analysis quality module 310 applies analysis quality rules. In one embodiment, the quality analysis rules check for features in the data that may indicate a high likelihood of inaccurate results. For example, one such rule might compare the total area for a chromatogram to a minimum area threshold and flag the test data as low-quality if the total area is less than the threshold. If the test data is flagged as low-quality data, the analysis quality module 310 may end the analysis and indicate that a new test should be performed. This prevents time and resources being wasted on further analysis of data that is unreliable. In such cases, the analysis quality module 310 may automatically trigger retesting of the sample. Another analysis quality rule might look at the width of a known peak (e.g., the A1c or A2 peaks) and add a warning comment if the width exceeds an expected width threshold. Other examples include rules checking for uneven baselines and highly asymmetrical peaks (e.g., peak tailing). In some embodiments, quality analysis rules may also be used to identify unusual result patterns that may require specialized analysis. For example, if a chromatogram does not include an A0 peak, this may indicate that the test data is unreliable or it may indicate that the subject is homozygote or double heterozygote. If all of the other peaks are within expected ranges, the analysis quality module 310 provides an alert of a potential system malfunction. In contrast, if another peak (e.g., greater than 60% of the total area) or two other peaks (e.g., both greater than 25% of the total area) are detected, the smart advisor 240 might add a note and/or comment suggesting a homozygous or a double heterozygous condition, respectively.

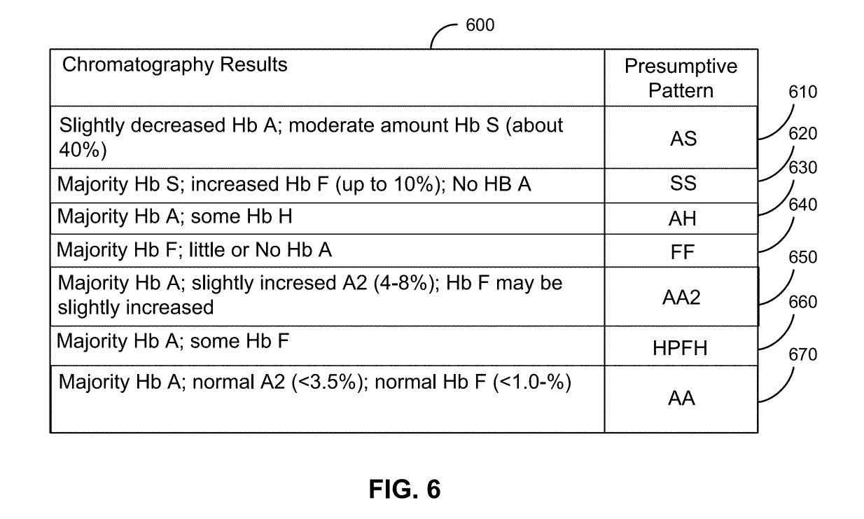

FIG. 6 is a table illustrating some example patterns, according to one embodiment. Presumptive patterns are a set of rules regarding the presence (or absence) and size of peaks that correspond to a particular condition. If the peaks in blood test data match the pattern, it indicates the blood may have come from an individual with the corresponding condition. For example, the first line 610 indicates that if the chromatography results show a slightly decreased amount of HbA (relative to a normal range) and a moderate amount of HbS (e.g., about 40%), then the presumptive pattern AS is selected. The second line 620 indicates that a majority of HbS coupled with an increased amount of HbF (up to 10%) and no HbA corresponds to presumptive pattern SS. The third through seventh lines 630, 640, 650, 660, and 670 provide the conditions that correspond to presumptive patterns AH, FF, AA2, HPFH, and AA, respectively. In other embodiments, additional or different rules are used to map observed chromatography results to presumptive patterns. Furthermore, as described previously, in some embodiments, the pattern rules can be modified or added to by authorized users, making the smart advisor 240 easily upgradable to reflect the latest information available (e.g., new discoveries published in academic literature, such as previously unknown conditions or correlations).

Referring back to FIG. 3, the variant identification module 320 applies variant identification rules to the input data (including any notes or comments added by analysis quality module 310) to classify peaks as normal or unusual and identify peaks that correspond to variants. In one embodiment, each peak has a pair of flags: an “unusual peak” flag and a “variant peak” flag. All variant peaks are also unusual, but the converse is not true. An unusual peak is one that appears in an unexpected location or has an unexpected area (e.g., if the hemoglobin F peak were higher than expected for a healthy adult, the unusual peak flag would be set). The variant peak flag indicates that a peak likely corresponds to a hemoglobin variant.

For example, one variant identification rule might state that an unknown peak with an area above an unknown peak threshold will be labelled as a variant. The size at which a peak will be labelled as a variant may depend on the window in which the peak appears. For example, a small peak in the S-window or the C-window may be labelled as a variant. In addition, a small increase or decrease in the size of an expected peak may also be labelled as unusual (e.g., raised HbF in a pregnant woman, raised HbF due to a hematological malignancy, decreased HbA2 levels due to iron deficiency anemia, raised HbA2 due to HIV therapy or hyperthyroidism, and the like). The variant identification module 320 may also relabel one or more peaks based on rules indicating that the original label is inaccurate.

The pattern comparison module 330 applies pattern rules to the input data as labelled by the variant identification module 320. The pattern comparison module 330 may also calculate special sums that combine data from one or more peaks to aid in efficient analysis. For example, a total hemoglobin A percentage (HbA) may be calculated by combining all pertinent peaks (e.g., A1a, A1b, P3, LA1c, A1c, and A0), with any peaks that were previously labelled as variants being omitted. As other examples, a more accurate total hemoglobin F (HbF) value can be obtained by summing the peaks for Acetylated HbF and “regular” HbF, and HbA2 and the variant, HbA2′. can be summed to reduce the risk of missing a co-inherited beta thalassemia.

In one embodiment, each pattern rule considers the area of one or more peaks to identify whether a corresponding preliminary pattern that matches the input data. If the conditions of the pattern rule are met, the rule is triggered and the pattern comparison module 330 adds an indicator of the associated preliminary pattern to the data. The pattern comparison module 330 may also add one or more comments. For example, the comments might identify potential diagnosis pitfalls related to the preliminary pattern (e.g., conditions with similar patterns that are often confused with each other), include quotes from and/or links to scientific literature regarding the corresponding condition, suggest further testing that would help reach a diagnosis (e.g., a follow-up test that will distinguish between two or more conditions with similar patterns), and/or identify other factors that should be considered (e.g., the ethnicity of the subject).

The results evaluation module 340 receives the output from the pattern comparison module 330 and applies results evaluation rules. The results evaluation rules provide overall analysis considering comments and flags added by earlier applied rules. For example, when testing for Beta Thalassemia, the generated report may include an HbA1c result as well as information about the hemoglobin pattern, along with associated comments and notes. This can enable a more complete assessment of the diabetes control in the presence of hemoglobinopathies that can alter red blood cell lifespan. The added comments may alert the laboratory scientist and help the clinician in the interpretation of the result.

In one embodiment, the results evaluation module 340 may set a flag indicating that the test results should be suppressed and/or repeated (e.g., if the analysis suggests the results are unreliable). The results evaluation module 340 may also add additional comments regarding features of the test results, such as the presence of a specific hemoglobin variant. The application of result evaluation rules after application of the other rule sets allows, among other things, the identification of patterns based on retention time, range percentages, and the like. It also may allow variant identifications to be customized based on the detection of known variants and/or the addition of comments and notes to each pattern.

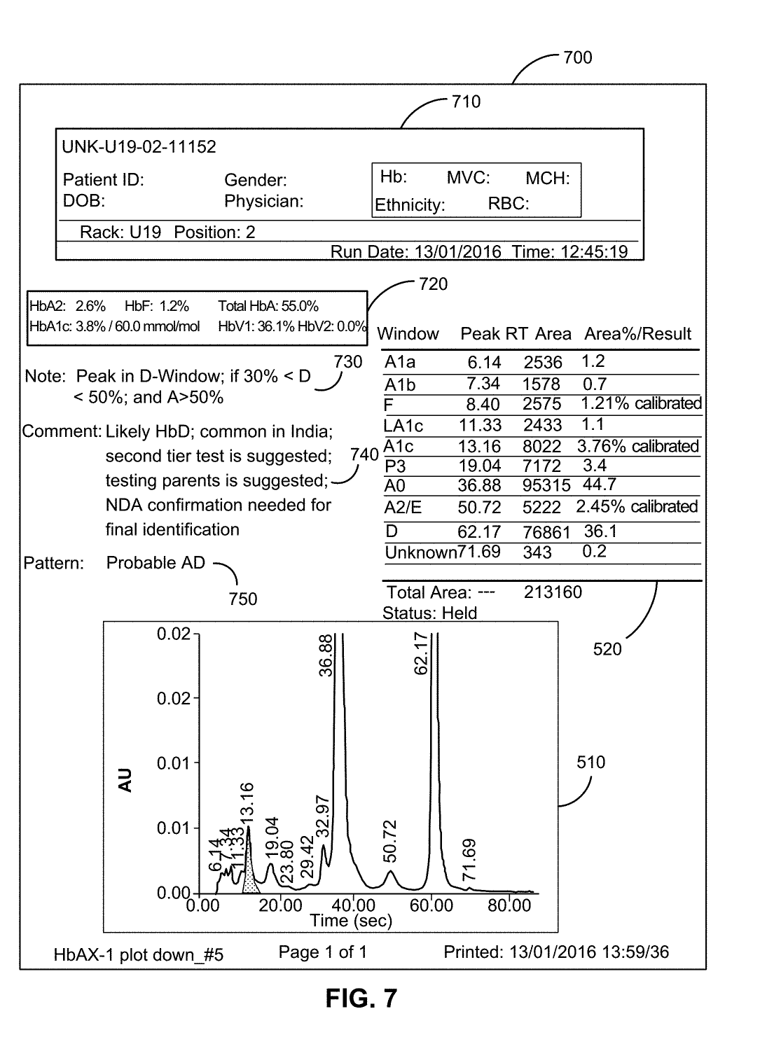

FIG. 7 shows an example enhanced report 700 generated by the smart advisor 240, according to one embodiment. The enhanced report 700 integrates the chromatogram with information collected from the LIS 110, such as hematology data (e.g., complete blood count results), ethnicity, pregnancy, reproductive status (age, partner being tested, etc.), which all together contribute to a more complete evaluation of the case. In the embodiment shown in FIG. 7, the enhanced report 700 includes the data table 520 and corresponding visual representation 510 that was included in the chromatogram 500. In addition, the enhanced report includes test information 710, some calculated special sums 720, one or more notes 730, one or more comments 740, and the preliminary pattern 750 selected by the smart advisor 240. In one embodiment, any comments generated by the smart advisor 240 are presented to a laboratory supervisor (e.g., at a terminal 130) and are only included on the enhanced report 700 if the laboratory supervisor approves them. In other embodiments, the enhanced report 700 includes different and/or additional information.

The test information 710 identifies the subject (e.g., with a patient ID) and provides other pertinent information, such as the responsible physician, demographic data, the date and time of the test, and the like. The calculated special sums 720 are those values that were calculated by the pattern comparison module 330. The notes 730 and comments 740 sections display any annotations added by rules by the smart advisor 240. As discussed previously, the preliminary pattern 750 is selected by the smart advisor 240 based on the application of the rules.

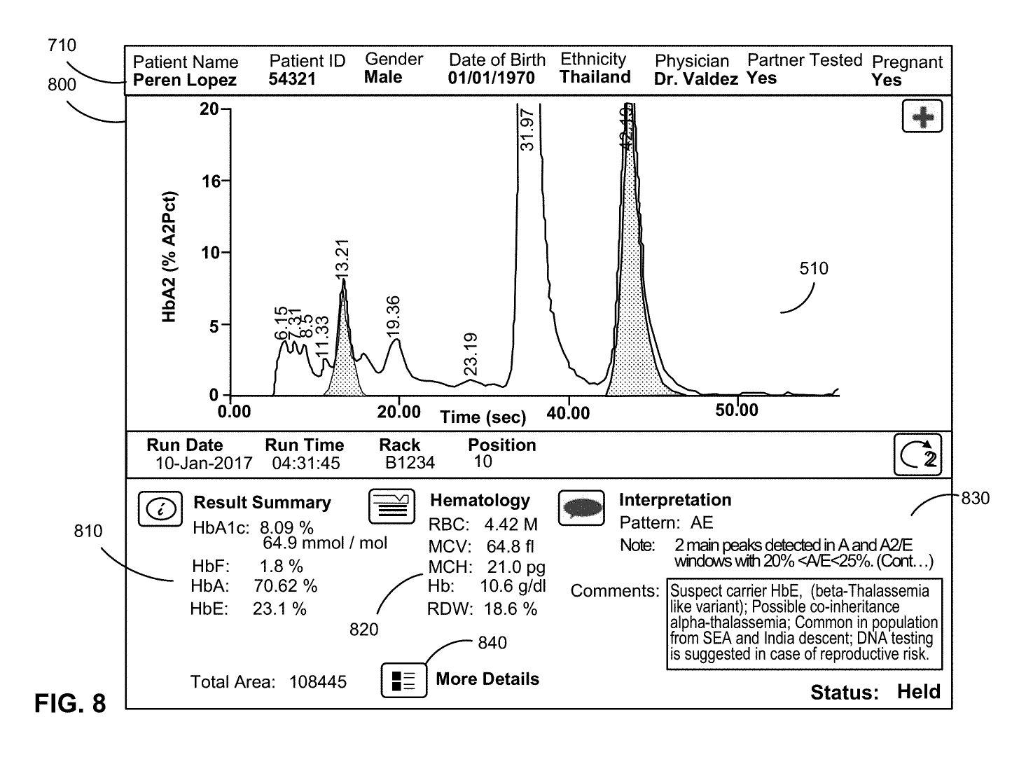

FIG. 8 shows an example of an enhanced report 800 generated by the smart advisor 240 and displayed on a terminal 130, according to one embodiment. The enhanced report 800 includes test information 710 and a visual representation of the data 510, similar to the enhanced report 700 shown in FIG.7. The enhanced report 800 of FIG. 8 also includes a results summary 810, hematology information 820, interpretation information 830, and a button 840 to request more details.

The results summary 810 includes the percentage of various hemoglobin variants that were present in the sample. This is a less detailed view of the data than was included in the enhanced report 700 of FIG. 7, and thus may be easier or faster to interpret. Note that the results summary 810 may reflect the name change for peaks that were renamed by the variant identification rules and/or include special sums (e.g., total HbA) calculated by the smart advisor 240. In other words, the summary 810 provides a quick overview to aid users in interpreting what the report 800 shows. The hematology 820 includes various data about the blood sample, such as the red blood cell count, mean corpuscular volume, and the like. The interpretation information 830 includes the preliminary pattern selected by the smart advisor 240 as well as any notes 730 and comments 740. User selection of the more details button 840 causes the terminal 130 to display additional information about the test. For example, the additional information might include configuration details, audit trail information, the specific rules used, quality metrics, and the like.

### Computing System Architecture

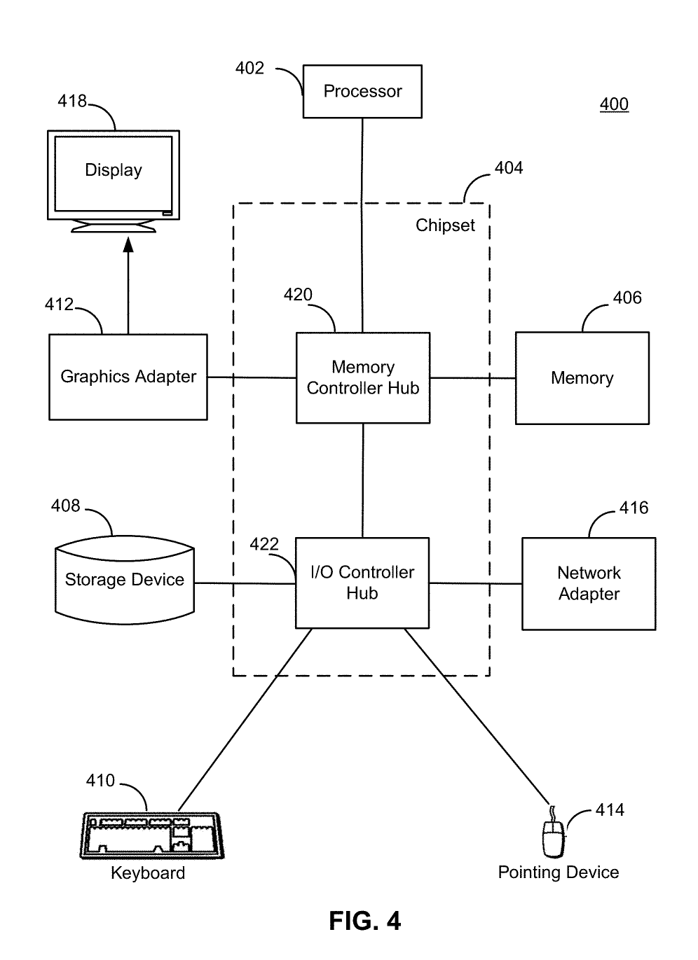

FIG. 4 illustrates an example computer 400 suitable for use as a laboratory terminal 120 or LIS 110, according to one embodiment. The example computer 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, keyboard 410, pointing device 414, and network adapter 416 are coupled to the I/O controller hub 422. Other embodiments of the computer 400 have different architectures.

In the embodiment shown in FIG. 4, the storage device 408 is a non-transitory computer-readable storage medium such as a hard drive, compact disk read-only memory (CD-ROM), DVD, or a solid-state memory device. The memory 406 holds instructions and data used by the processor 402. The pointing device 414 is a mouse, track ball, touch-screen, or other type of pointing device, and is used in combination with the keyboard 410 (which may be an on-screen keyboard) to input data into the computer system 400. The graphics adapter 412 displays images and other information on the display 418. The network adapter 416 couples the computer system 400 to one or more computer networks.

The types of computers used by the entities of FIGS. 1 through 3 can vary depending upon the embodiment and the processing power required by the entity. For example, an LIS 110 might include a distributed database system comprising multiple blade servers working together to provide the functionality described. Furthermore, the computers can lack some of the components described above, such as keyboards 510, graphics adapters 512, and displays 518.

### Example Methods

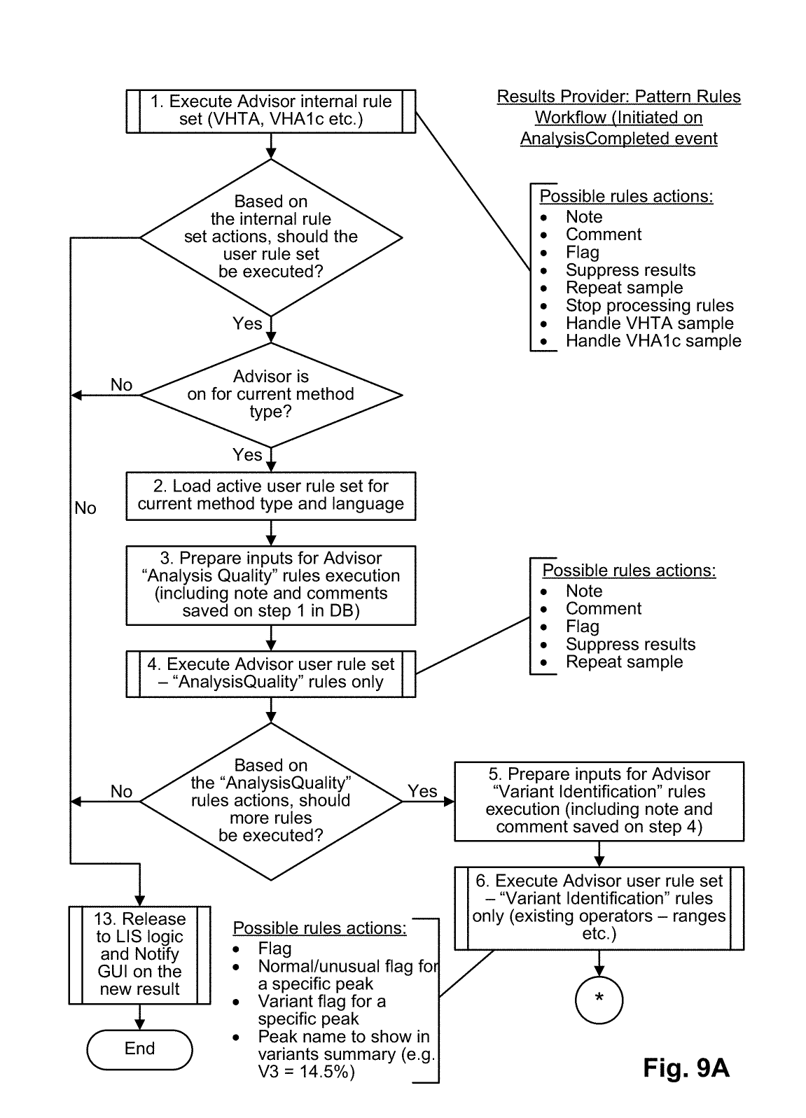

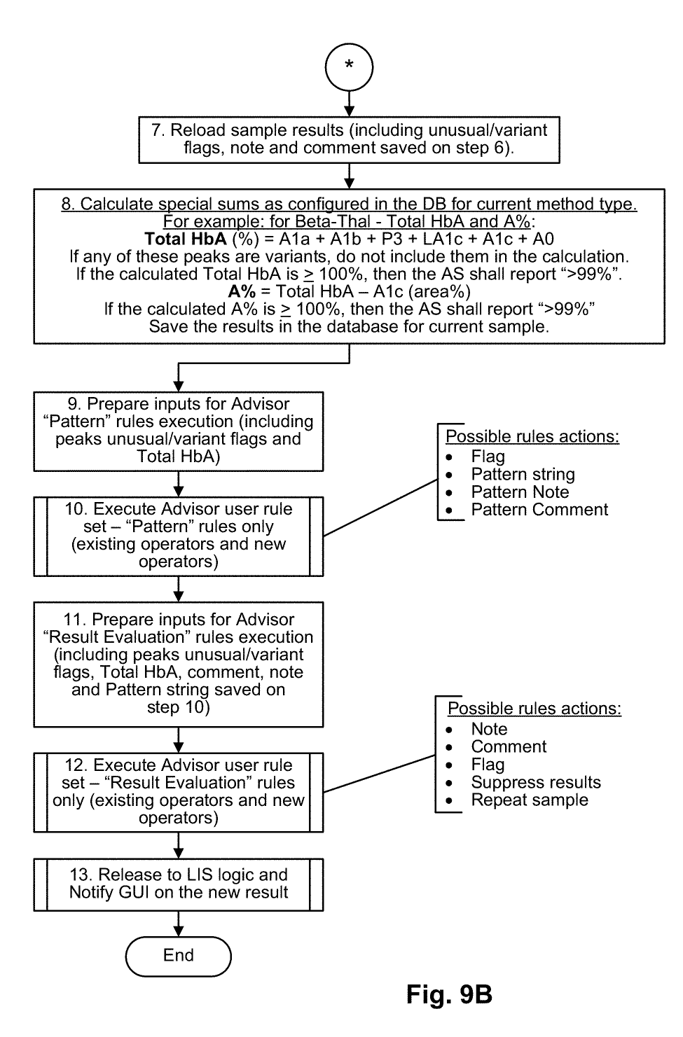

FIGS. 9A and 9B illustrate a method for producing an enhanced report (e.g., smart report 700), according to one embodiment. The steps of FIGS. 9A and 9B are illustrated from the perspective of the smart advisor 240 performing the method. However, some or all of the steps may be performed by other entities or components. In addition, some embodiments may perform the steps in parallel, perform the steps in different orders, or perform different steps.

In the embodiment shown, the method begins with the smart advisor 240 executing an internal rule set. The internal rule set can result in the smart advisor 240 adding notes, comments, and/or flags to the data set. The internal rules can also cause the smart advisor to suppress the results, instruct that lab equipment 120 should repeat a test on the sample, stop processing of the sample, and/or treat the sample as a VHTA or VHA1c sample.

If the internal rule set indicates that analysis should continue, the smart advisor 240 checks whether the current method type has the smart advisor activated. Assuming so, the smart advisor loads the user rule set for the current method and language (e.g., from local storage 260). The smart advisor 240 then prepares inputs for a set of analysis quality rules (within the user rules) from the data set, the inputs including notes and comments added due to the internal rules. The smart advisor 240 then applies the analysis quality rules, adding notes, comments, and flags as appropriate, and instructing that the results be suppressed or the sample test repeated if required. If the analysis quality rule set is empty (i.e., there are no such rules for the current method), the smart advisor proceeds to application of any variant identification rules.

Unless application of the analysis quality rules indicates that the analysis should be terminated, the smart advisor 240 prepares the input for and applies a set of variant identification rules. As described previously, these rules identify peaks in the data set and set normal/unusual flags and variant flags as indicated by the rules. The variant identification rules may also rename certain peaks.

The smart advisor 240 uses the data set, including the flags set by the variant identification rules, to calculate any special sums defined for the method. The smart advisor 240 then prepares inputs for and applies a set of pattern rules. As described previously, these rules identify a presumptive pattern that matches the data set. These rules can also add flags, pattern notes, and pattern comments to the data set.

The smart advisor 240 also prepares inputs for and applies a set of result evaluation rules. These rules can add additional notes, comments, and flags to the data set, as well as instruct that the results be suppressed or the sample testing repeated.

At whatever point the analysis of the smart advisor 240 ends, whether due to reaching the end of the rules chain or because one of the rule sets indicated that analysis should be ended early (e.g., because the test results were unreliable), the results are released to the LIS 110 and the user is notified via the user interface on the terminal 130. Note that is one of the rule sets determines that the results should be suppressed, they may not be released to the LIS 110.

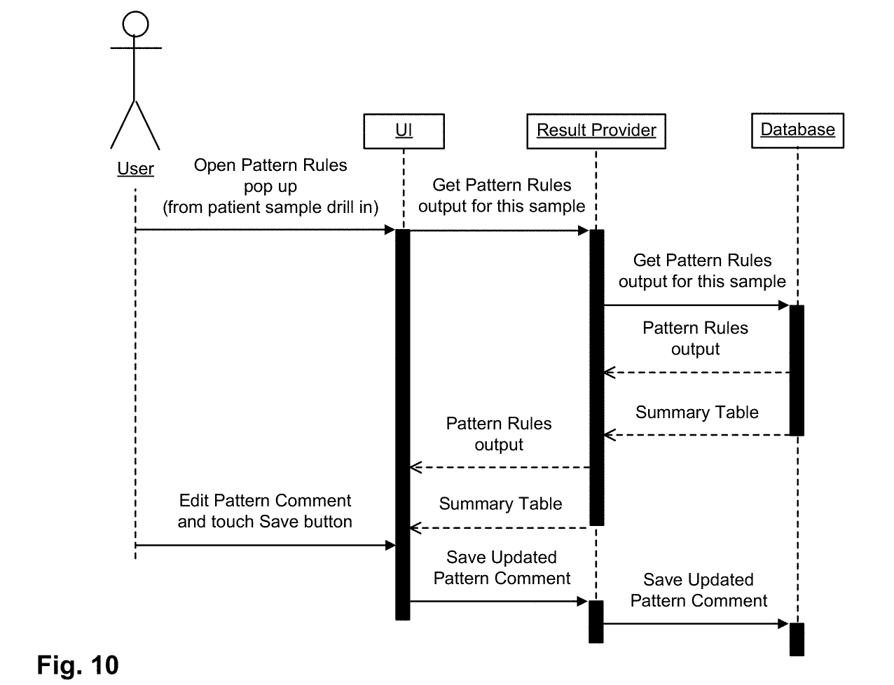

FIG. 10 is an interactions diagram illustrating a drill in operation to edit the comments of a pattern rule, according to one embodiment. In the embodiment shown in FIG. 10, a user uses a user interface (e.g., on a terminal 130) to enter a patient sample drill in and opens the pattern comments editor (e.g., a pattern rules pop-up). The user interface requests the pattern rules output for the current sample from a result provider, which in turn retrieves the output along with a summary table from a database. The requested patterns rules output and the summary table are provided to the user interface, which presents them to the user. The user enters edits to the pattern comments and submits them from the user interface (e.g., by pressing a save button). The user interface sends the updated comments to the results provider that updates the comments stored in the database. In other embodiments, other methods of editing pattern comments are provided.

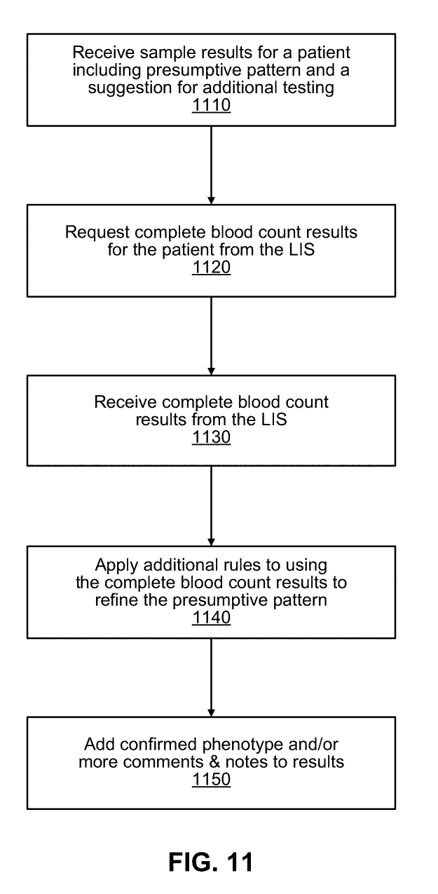

FIG. 11 is a flow-chart illustrating a method for generating an enhanced report that includes automatically obtaining additional test data to refine the presumptive pattern identified in the enhanced report, according to one embodiment. The steps of FIG. 11 are illustrated from the perspective of the smart advisor 240 performing the method, for example, with pattern refining module (not shown). However, some or all of the steps may be performed by other entities or components. In addition, some embodiments may perform the steps in parallel, perform the steps in different orders, or perform different steps.

In the embodiment shown in FIG. 11, the method begins with the smart advisor 240 receiving 1110 sample results for a patient including a presumptive pattern and a suggestion for additional testing. For example, the sample results may have been generated by the smart advisor 240 using the method described above with reference to FIGS. 9A and 9B.

The smart advisor 1120 requests 1120 complete blood count results for the patient from the LIS 110. In response, the LIS 110 returns the requested results, which are then received 1130 by the smart advisor 240 (assuming the requested results are available). The smart advisor 240 applies 1140 additional rules using the received complete blood count results to refine the presumptive pattern. The smart advisor then adds 1150 additional information to the enhanced report based on the application of the additional rules. For example, if the complete blood count results confirm that the presumptive pattern is correct (rather than a similar pattern that may be confused with the presumptive pattern), the presumptive pattern might be marked as confirmed. As another example, if analysis based on the complete blood count results is still inconclusive, a comment suggestion another kind of testing (e.g., DNA testing of relatives) might be added. One of skill in the art will recognize various comments and notes that may be added to the report based on the complete blood count results.

### Example Results and Interfaces

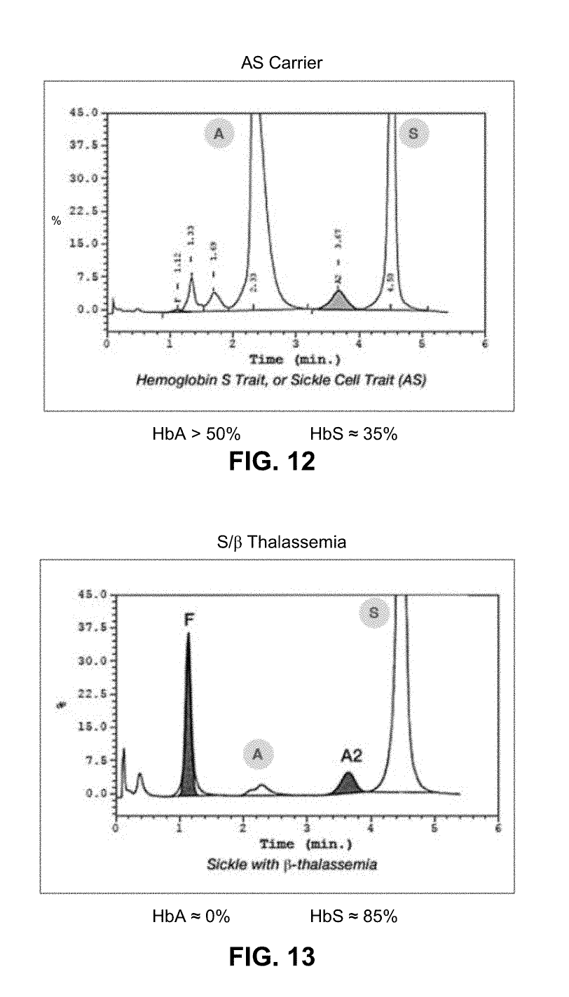

FIG. 12 is a plot of example blood test data for an individual who is a carrier of the sickle cell S trait (an “AS carrier”). Blood test data for an AS carrier may be characterized as having an HbA peak over 50% and an HbS peak of approximately 35%. Thus, in one embodiment, a smart advisor rule may states that if the blood test data includes an HbA peak of over 50% and an HbS peak of approximately 35% (e.g., 30-40%, 32-38%, 34-36%, etc.) then the possibility that the individual is an AS carrier is added to the resulting smart report along with information about the condition, suggested follow-up tests, links to literature with additional information, and the like.

FIG. 13 is a plot of example blood test data dataset for an individual who has sickle cell with β thalassemia. Blood test data for such an individual may be characterized as having an HbA peak of approximately 0% (i.e., a very small peak or no peak at all) and an HbS peak of approximately 85%. Thus, in one embodiment, a smart advisor rule may states that if the blood test data includes an HbA peak of approximately 0% (e.g., under 5%, under 2%, under 1% etc.) and an HbS peak of approximately 85% (e.g., 80-90%, 82-88%, 84-86%, etc.) then the possibility that the individual has sickle cell with β thalassemia is added to the resulting smart report along with information about the condition, suggested follow-up tests, links to literature with additional information, and the like.

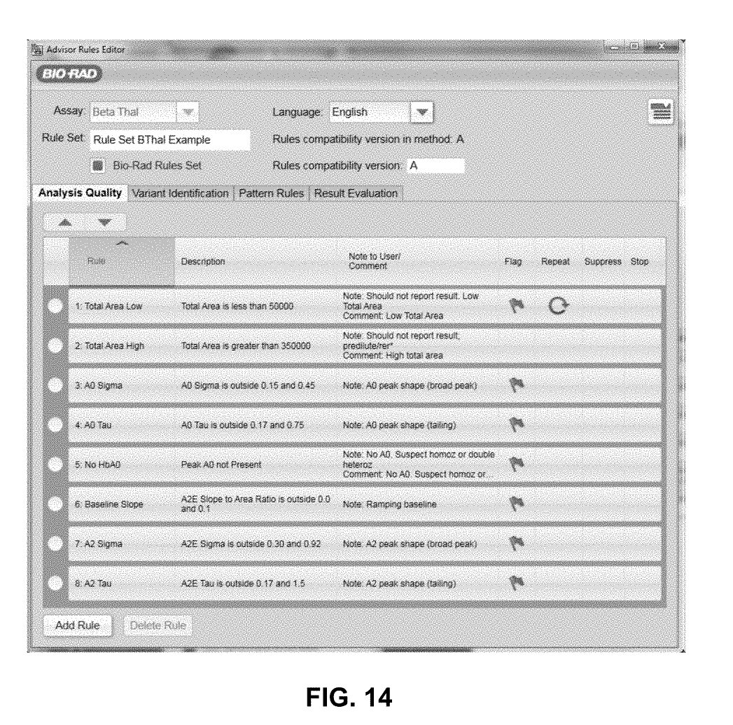

FIG. 14 is a screenshot illustrating analysis quality rules in a rule editor, according to one embodiment. The analysis quality rules provide an indication of the quality of the results obtained from a blood sample. For example, if the total area for a result is too low or high, this may indicate that the amount of blood in the test sample was either too low or too high. Thus, a warning may be added to a generated report, such as a recommendation to re-run the test along with suggestions as to what might have caused the problem. In some embodiments, analysis quality rules may trigger automatic retesting of a sample.

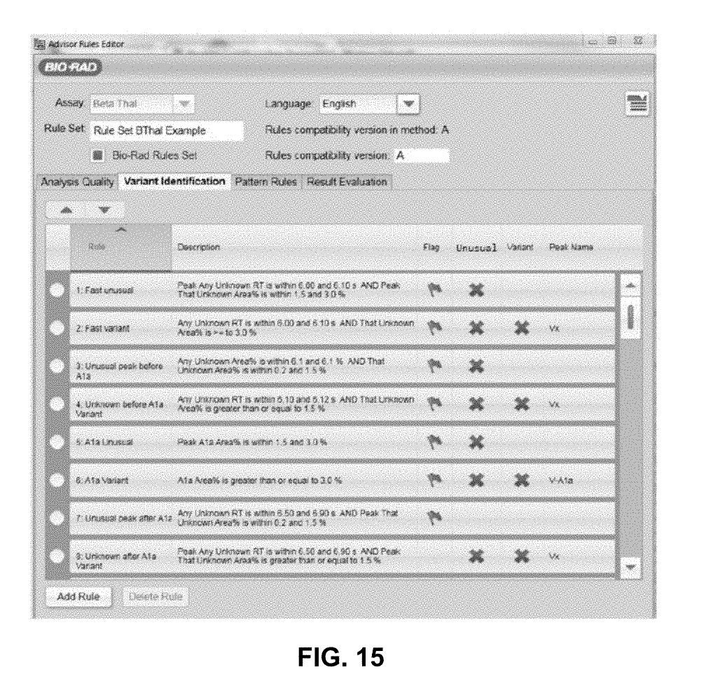

FIG. 15 is a screenshot illustrating variant identification rules in a rule editor, according to one embodiment. The variant identification rules identify peaks that are variants of typical peaks, at unusual locations (e.g., unknown peaks), and the like. For example, the first variant identification rule shown causes any unknown peak with an area between 1.5% and 3.0% within the range from 6.00 seconds to 6.10 seconds to be labelled as a “fast unusual” peak. Similarly, the second rule causes any unknown peak with an area greater than or equal to 3.0% within the range from 6.00 seconds to 6.10 seconds to be labelled as a “fast variant” peak. These rules may also determine whether such peaks should be flagged as just unusual or both unusual and variant. For example, the first rule labels fast unusual peaks as unusual but not a variant while the second rule labels fast variant peaks as both unusual and a variant.

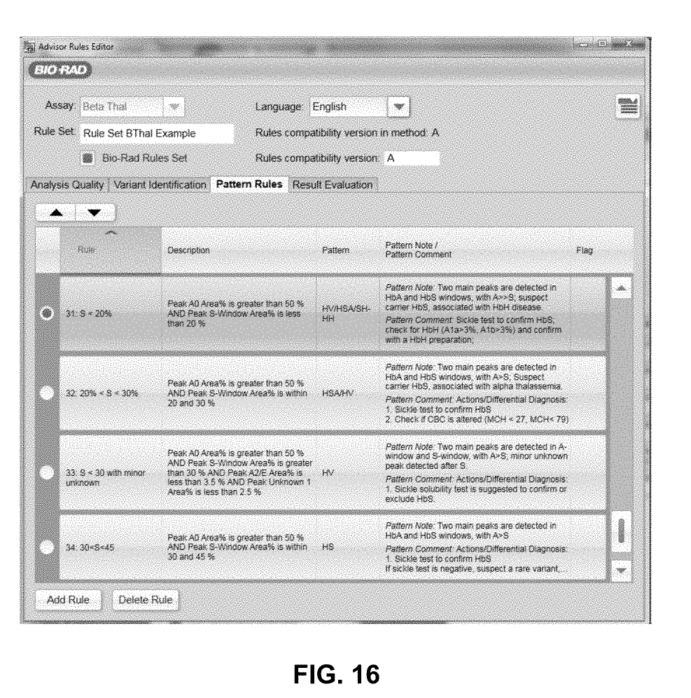

FIG. 16 is a screenshot illustrating pattern rules in a rule editor, according to one embodiment. The pattern rules define criteria and assign a pattern (corresponding to a condition) to results if the defined criteria are met. The for example, the first pattern rule shown causes blood test result where the A0 peak area is greater than 50% and the Peak-S window area is less than 20% to be labelled with the HV/HAS/SH-HH pattern. The pattern rules may also define comments and/or notes regarding the pattern that are added to the enhanced report. For example, the first rule shown includes a note summarizing the peaks detected that led to the pattern being selected and possible conditions that might result in those peaks. The first rule shown also includes comment recommending further testing to be performed to assist in accurate diagnosis.

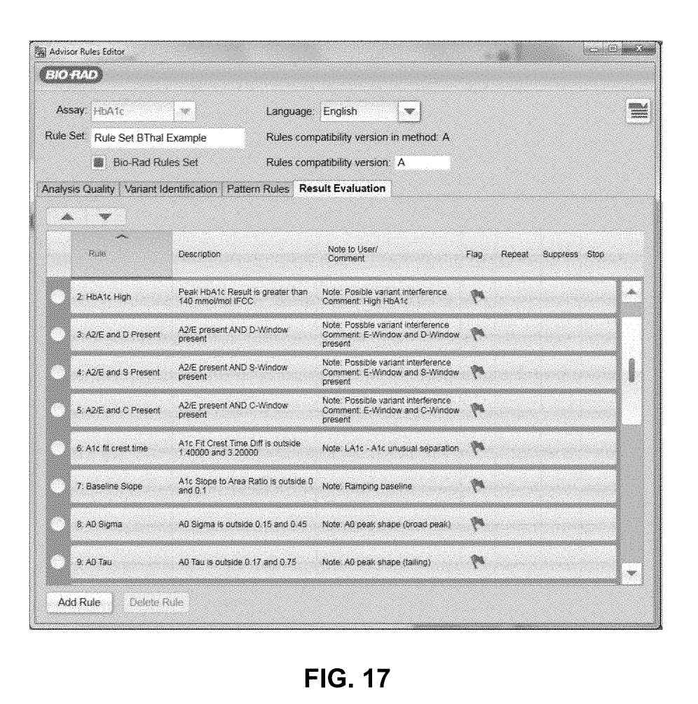

FIG. 17 is a screenshot illustrating result evaluation rules in a rule editor, according to one embodiment. The result evaluation rules provide additional analysis that may incorporate labels added by some or all of the previously applied rules. For example, the first rule shown adds a note to the enhanced report if the HbA1c peak exceeds a threshold indicating that the may be interference in the result due to a variant. The rule also adds a comment explaining why the note was included (in this case, the unusually high HbA1c peak).

### Additional Considerations

Some portions of above description describe the embodiments in terms of algorithmic processes or operations. These algorithmic descriptions and representations are commonly used by those skilled in the data processing arts to convey the substance of their work effectively to others skilled in the art. These operations, while described functionally, computationally, or logically, are understood to be implemented by computer programs comprising instructions for execution by a processor or equivalent electrical circuits, microcode, or the like. Furthermore, it has also proven convenient at times, to refer to these arrangements of functional operations as modules, without loss of generality.

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments. This is done merely for convenience and to give a general sense of the disclosure. This description should be read to include one or at least one and the singular also includes the plural unless it is obvious that it is meant otherwise.

Upon reading this disclosure, those of skill in the art will appreciate still additional alternative structural and functional designs for a system and a process for providing a smart advisor that aids in hemoglobinopathy evaluation. Thus, while particular embodiments and applications have been illustrated and described, it is to be understood that the described subject matter is not limited to the precise construction and components disclosed herein and that various modifications, changes and variations which will be apparent to those skilled in the art may be made in the arrangement, operation and details of the method and apparatus disclosed. The scope of protection should be limited only by the following claims.

What is claimed is:

1. A method for generating an enhanced report from blood test data, the method comprising:
receiving blood test chromatography data for a blood sample of a patient, the data including a plurality of peaks, each peak of the plurality of peaks corresponding to a type of hemoglobin and having a value indicating an amount of the corresponding type of hemoglobin present in the blood sample;
applying a set of variant identification rules to identify at least one of the peaks as an abnormal peak;
applying a set of pattern rules to identify a presumptive pattern indicative of a medical condition, the presumptive pattern identified based on the abnormal peak;
generating the enhanced report based on the presumptive pattern, the enhanced report including at least one comment providing advice regarding interpretation of the enhanced report; and
providing the enhanced report for display at a terminal.

2. The method of claim 1, further comprising applying a set of analysis quality rules to determine an indication of quality of the blood test data.

3. The method of claim 1, further comprising applying a set of result evaluation rules to determine at least some of the advice regarding interpretation of the enhanced report.

4. The method of claim 1, wherein the advice regarding interpretation of the enhanced report includes at least one of: a common pitfall associated with the presumptive pattern, a recommendation for additional testing, or additional information about the medical condition.

5. The method of claim 1, further comprising calculating a special sum from the blood test chromatography data, the special sum combining data from a plurality of peaks.

6. The method of claim 5, wherein the special sum is a total hemoglobin A amount, calculating the special sum comprising:
identifying a subset of the plurality of peaks that correspond to variants of hemoglobin A;
determining, for each peak in the subset, whether that peak was identified as abnormal; and
summing the value of each peak in the subset that was not identified as abnormal.

7. The method of claim 1, wherein the enhanced report includes a recommendation for additional testing, the method further comprising:
requesting complete blood count results for the patient from a database;
receiving the requested complete blood count results;
applying a set of additional rules to refine the presumptive pattern based on the complete blood count results; and
updating the enhanced report based on the refinement.

8. A computer-based system for generating an enhanced report from blood test data, the system comprising:
one or more processors; and
a computer readable medium storing computer program code that, when executed, causes the one or more processors to perform operations including:
receiving blood test chromatography data for a blood sample of a patient, the data including a plurality of peaks, each peak of the plurality of peaks corresponding to a type of hemoglobin and having a value indicating an amount of the corresponding type of hemoglobin present in the blood sample;
applying a set of variant identification rules to identify at least one of the peaks as an abnormal peak;
applying a set of pattern rules to identify a presumptive pattern indicative of a medical condition, the presumptive pattern identified based on the abnormal peak;
generating the enhanced report based on the presumptive pattern, the enhanced report including at least one comment providing advice regarding interpretation of the enhanced report; and
providing the enhanced report for display at a terminal.

9. The system of claim 8, wherein the operations further comprise applying a set of analysis quality rules to determine an indication of quality of the blood test data.

10. The system of claim 8, wherein the operations further comprise applying a set of result evaluation rules to determine at least some of the advice regarding interpretation of the enhanced report.

11. The system of claim 8, wherein the advice regarding interpretation of the enhanced report includes at least one of: a common pitfall associated with the presumptive pattern, a recommendation for additional testing, or additional information about the medical condition.

12. The system of claim 8, wherein the operations further comprise calculating a special sum from the blood test chromatography data, the special sum combining data from a plurality of peaks.

13. The system of claim 12, wherein the special sum is a total hemoglobin A amount, calculating the special sum comprising:
identifying a subset of the plurality of peaks that correspond to variants of hemoglobin A;
determining, for each peak in the subset, whether that peak was identified as abnormal; and
summing the value of each peak in the subset that was not identified as abnormal.

14. The system of claim 8, wherein the enhanced report includes a recommendation for additional testing, and the operations further comprise:
requesting complete blood count results for the patient from a database;
receiving the requested complete blood count results;
applying a set of additional rules to refine the presumptive pattern based on the complete blood count results; and
updating the enhanced report based on the refinement.

15. A non-transitory computer-readable medium storing computer program instructions executable by a processor to perform operations comprising:
receiving blood test chromatography data for a blood sample of a patient, the data including a plurality of peaks, each peak of the plurality of peaks corresponding to a type of hemoglobin and having a value indicating an amount of the corresponding type of hemoglobin present in the blood sample;
applying a set of variant identification rules to identify at least one of the peaks as an abnormal peak;
applying a set of pattern rules to identify a presumptive pattern indicative of a medical condition, the presumptive pattern identified based on the abnormal peak;
generating an enhanced report based on the presumptive pattern, the enhanced report including at least one comment providing advice regarding interpretation of the enhanced report; and
providing the enhanced report for display at a terminal.

16. The non-transitory computer-readable medium of claim 15, wherein the operations further comprise applying a set of analysis quality rules to determine an indication of quality of the blood test data.

17. The non-transitory computer-readable medium of claim 15, wherein the operations further comprise applying a set of result evaluation rules to determine at least some of the advice regarding interpretation of the enhanced report.

18. The non-transitory computer-readable medium of claim 15, wherein the advice regarding interpretation of the enhanced report includes at least one of: a common pitfall associated with the presumptive pattern, a recommendation for additional testing, or additional information about the medical condition.

19. The non-transitory computer-readable medium of claim 15, wherein the operations further comprise calculating a total hemoglobin A amount, calculating the total hemoglobin A amount comprising:
identifying a subset of the plurality of peaks that correspond to variants of hemoglobin A;
determining, for each peak in the subset, whether that peak was identified as abnormal; and
summing the value of each peak in the subset that was not identified as abnormal.

20. The non-transitory computer-readable medium of claim 15, wherein the enhanced report includes a recommendation for additional testing, the operations further comprising:
requesting complete blood count results for the patient from a database;
receiving the requested complete blood count results;
applying a set of additional rules to refine the presumptive pattern based on the complete blood count results; and
updating the enhanced report based on the refinement.
